(19) United States
(12) Patent Application Publication
(10) Pub. No.: US 2012/0253447 A1
(43) Pub. Date: Oct. 4, 2012

(54) BALLOON CATHETER

(75) Inventors: Kazuma HAYASAKA, Ashigarakami-gun (JP); Michiyo TSUBOOKA, Ashigarakami-gun (JP); Tatsurou OOYA, Ashigarakami-gun (JP)

(73) Assignee: TERUMO KABUSHIKI KAISHA, Shibuya-ku (JP)

(21) Appl. No.: 13/523,666

(22) Filed: Jun. 14, 2012

Related U.S. Application Data
(63) Continuation of application No. PCT/JP2010/072089, filed on Dec. 9, 2010.

(30) Foreign Application Priority Data
Dec. 28, 2009 (JP) 2009-297116

(51) Int. Cl. A61M 25/10; A61F 2/84

(52) U.S. Cl. 623/1.11; 606/194

(57) ABSTRACT

A balloon catheter includes: an inner tube adapted to receive a guide wire; an outer tube disposed on the outer surface side of the inner tube and cooperating with the inner tube to define an inflation lumen therebetween; and a balloon joined at the distal end thereof to the inner tube and at the proximal end thereof to the outer tube and configured to be expanded by inflation fluid from the inflation lumen. A non-circular region, having a non-circular sectional shape taken in a direction orthogonal to an extending direction of the inner tube, is formed on at least one of an outer surface of the inner tube and an inner surface of the outer tube. The non-circular region at least includes a joint section between the balloon and the outer tube and extends proximally of the joint section.

This application is a continuation of International Application No. PCT/JP2010/072089 filed on Dec. 9, 2010, and claims priority to Japanese Application No. 2009-297116 filed on Dec. 28, 2009, the entire content of both of which is incorporated herein by reference.

### TECHNICAL FIELD

The present invention generally relates to a balloon catheter for use in treatments and the like of a stenosis in a biological organ.

### BACKGROUND DISCUSSION

BY way of example, treatment of myocardial infarction or angina can involve the use of a method in which a lesion (stenosis) of a coronary artery is expanded by a balloon catheter. A similar method is sometimes used also for improving a stenosis in a biological organ such as other blood vessels, a bile duct, a trachea, an esophagus, a urethra or other organs.

The balloon catheter generally includes an elongated shaft main body and a balloon disposed at a distal end of the shaft main body and adapted to expand in the radial direction and is positioned at a stenosis in a body through use of a guide wire. Then, by delivering inflation fluid to an inflation lumen in the shaft main body by way of an indeflator or the like in a state in which the balloon is disposed at a stenosis which is a target region, the balloon is expanded so that the stenosis can be expanded.

A balloon catheter in which an inflation lumen is formed between an inner tube into which the guide wire is to be inserted and an outer tube provided on an outer periphery side of the inner tube is disclosed in Japanese Patent Laid-Open No. Hei 7-265437. In Japanese Patent Laid-Open No. Hei 7-265437, in order to smoothly advance the balloon catheter to a meandering stenosis or the like, a configuration is disclosed in which, with the aim of enhancing flexibility of a distal end portion of the catheter, a concave portion and a spiral extending in an axial direction or a slit extending in a direction orthogonal to the axial direction is formed in the inner tube from the most distal end of the catheter to a predetermined position.

As shown in FIG. 13A, in a general balloon catheter, a shaft main body 102 is configured as a double pipe including an inner tube 104 and an outer tube 106, with an inflation lumen 108 between the inner tube 104 and the outer tube 106. Accordingly, in order to expand the balloon with certainty at a stenosis and then contract the balloon with certainty to remove the catheter from the body, it is necessary for the inflation fluid to be capable of circulating smoothly in the inflation lumen 108 upon expansion and contraction of the balloon.

However, with such a balloon catheter as described above, if the inner tube 104 or the outer tube 106 is elongated by tensile force or bending force upon insertion into the body, then the inner tube 104 and the outer tube 106 are brought into contact with each other to block the inflation lumen 108 as shown in FIG. 13B, resulting in the possibility that smooth expansion or contraction of the balloon may become difficult.

Further, where a balloon catheter is used for stent delivery in which a stent is disposed on an outer circumferential surface of a balloon, the balloon and a neighboring portion are acted upon by compulsory contractive force by the mounted stent. As a result, there is the possibility that an outer tube may be contracted to block an inflation lumen thereby to make not only smooth expansion or contraction of the balloon but also uniform expansion of the stent difficult.

### SUMMARY

The balloon catheter disclosed here includes: an inner tube configured to receive a guide wire; an outer tube disposed on the outer surface side of the inner tube and cooperating with the inner tube to define an inflation lumen between the inner tube and the outer tube; and a balloon having a distal end joined to the inner tube and a proximal end joined to the outer tube and expandable by inflation fluid from the inflation lumen. A non-circular region, having a non-circular sectional shape taken in a direction orthogonal to an extending direction of the inner tube, is formed on at least one of the outer surface of the inner tube and the inner surface of the outer tube. The non-circular region at least includes a joint section between the balloon and the outer tube in the extending direction of the inner tube and extends to a position on the proximal end side farther than the joint section.

In the balloon catheter having the configuration described above, the non-circular region extending in the extending direction of the inner tube is formed on at least one of the outer surface of the inner tube and the inner surface of the outer tube. Therefore, even when the balloon catheter is acted upon by tensile force and thereupon the outer tube or the like is elongated until the outer surface of the inner tube and the inner surface of the outer tube are brought into contact with each other, the inner tube and the outer tube are prevented from being closely contacted with each other over the whole circumference thereof. Therefore, circulation of inflation fluid in the inflation lumen can be ensured with certainty. Because the non-circular region includes the joint section between the balloon and the outer tube and extends to the position on the proximal end side farther than the joint section, the non-circular region is disposed at the region corresponding to the joint section which is estimated to be most likely to be blocked in the entire inflation lumen. Therefore, the inflation lumen can be prevented from being blocked with a higher degree of certainty.

Preferably, in the non-circular region, projecting portions are formed on at least one of the outer surface of the inner tube and the inner surface of the outer tube so as to project toward the other one of the inner surface and the outer surface and extend in the extending direction of the inner tube. With such a configuration, even when the balloon catheter is acted upon by tensile force and the outer tube or the like is elongated, for example, since the projecting portions formed on the outer surface of the inner tube are abutted with the inner surface of the outer tube and a space is ensured around the projecting portions, the inflation lumen formed between the outer surface and the inner surface can be prevented from being blocked.

The balloon catheter may be configured such that, in the non-circular region, grooved portions are formed on at least one of the outer surface of the inner tube and the inner surface of the outer tube so as to extend in the extending direction of the inner tube. The non-circular region can also extend to a position which reaches an expansion portion of the balloon so that the inflation lumen can be prevented from being blocked with a higher degree of certainty.

A stent can be disposed on the outer circumferential surface of the balloon. In such a case, the non-circular region extends to a position including a region at which the stent is disposed in the extending direction of the inner tube. In that way, even if the balloon or the outer tube or the like around the balloon is acted upon by compulsory contraction by the stent and the outer tube is smashed, the inflation lumen can be prevented from being blocked with certainty. Consequently, uniform expansion of the stent can be anticipated together with smooth expansion or contraction of the balloon.

In the non-circular region, the sectional shape, in a direction orthogonal to the extending direction of the inner tube, of at least one of the outer surface of the inner tube and the inner surface of the outer tube may be a polygonal shape, a star shape or a shape having one or more concave portions.

Further, each of the grooved portions may have a spiral shape formed on at least one of the outer surface of the inner tube and the inner surface of the outer tube.

According to another aspect, a balloon catheter comprises: an elongated outer tube extending in a longitudinal direction and possessing a distal end portion, a proximal end portion and an intermediate portion located between the distal end portion and the proximal end portion; an inner tube located inside the outer tube and possessing an open distal end, an open proximal end and a lumen extending between the open distal end and the open proximal end, with the open proximal end of the inner tube communicating outside the outer tube at the intermediate portion of the outer tube, and the lumen in the inner tube being configured to receive a guide wire extending along the lumen and passing through the open distal end and the open proximal end; a balloon possessing a distal end portion fixed to the distal end portion of the inner tube and possessing a proximal end portion fixed to the distal end portion of the outer tube at an axially overlapping joint section at which the distal end portion of the outer tube and the proximal end portion of the bal loon axially overlap each other; and an inflation lumen between the inner surface of the outer tube and the outer surface of the inner tube to receive the inflation fluid to expand the balloon. A longitudinally extending portion of the inflation lumen is a non-circular region at which at least one of the outer surface of the inner tube and the inner surface of the outer tube possesses a non-circular cross-sectional shape in a direction perpendicular to the longitudinal axis of the outer tube, with the joint section located in the non-circular region of the inflation lumen, and the non-circular region of the inflation lumen extending proximally beyond a proximal-most end of the axially extending joint section.

In accordance with another aspect, a balloon catheter comprises: an elongated outer tube possessing a distal end portion, a proximal end portion and an intermediate portion located between the distal end portion and the proximal end portion, with the outer tube possessing an inner surface cross-sectional shape in a direction perpendicular to the longitudinal axis of the outer tube; an inner tube located inside the outer tube and possessing an outer surface cross-sectional shape in a direction perpendicular to the longitudinal axis of the outer tube, an open distal end, an open proximal end and a lumen extending between the open distal end and the open proximal end, with the proximal end portion of the inner tube passing through the tube wall of the outer tube at the intermediate portion of the outer tube so that the open proximal end of the inner tube communicates outside the outer tube at the intermediate portion of the outer tube, and wherein the lumen in the inner tube is configured to receive a guide wire extending along the lumen and passing through the open distal end and the open proximal end; and a balloon possessing a distal end portion fixed to the distal end portion of the inner tube and possessing a proximal end portion fixed to the distal end portion of the outer tube at an axially overlapping joint section at which the distal end portion of the outer tube and the proximal end portion of the balloon axially overlap each other. The balloon is configured to be expanded upon introducing an inflation fluid into the balloon and to be contractable when the inflation fluid is expelled from the balloon. An inflation lumen exist between the inner surface of the outer tube and the outer surface of the inner tube to receive the inflation fluid to expand the balloon. A longitudinally extending portion of the inflation lumen is a non-circular region at which: i) a size and a shape of the inner surface cross-sectional shape of the outer tube differ from a size and a shape of the outer surface cross-sectional shape of the inner tube; or ii) the size of the inner surface cross-sectional shape of the outer tube differs from the size of the outer surface cross-sectional shape of the inner tube, the shape of the inner surface cross-sectional shape of the outer tube is the same as the shape of the outer surface cross-sectional shape of the inner tube, and the inner surface cross-sectional shape of the outer tube is rotationally offset from the outer surface cross-sectional shape of the inner tube. The joint section is located in the non-circular region of the inflation lumen, and the non-circular region of the inflation lumen extending proximally beyond a proximal-most end of the axially extending joint section.

### BRIEF DESCRIPTION OF THE DRAWINGS

FIG. 1 is a general schematic view of a balloon catheter according to a first embodiment disclosed here.

FIG. 2 is an enlarged schematic view of a distal end side of the balloon catheter shown in FIG. 1.

FIG. 3A is a cross-sectional view taken along the section line WA-WA in FIG. 2, and FIG. 3B is a cross-sectional view showing an outer tube of the balloon catheter in a smashed state from the state illustrated in FIG. 3A.

FIG. 4A is a partially-omitted longitudinal cross-sectional view taken along an axial direction showing a state in which an inflation lumen of the balloon catheter is ensured by a grooved portion, and FIG. 4B is a similar view but showing another state in which the inflation lumen is not ensured even when the grooved portion is formed.

FIG. 5A is a cross-sectional view taken in a direction orthogonal to an axial direction of a grooved portion in a balloon catheter according to a first modified example, and FIG. 5B is a cross-sectional view showing an outer tube of the balloon catheter in a smashed state from the state illustrated in FIG. 5A.

FIG. 6A is a cross-sectional view taken in a direction orthogonal to an axial direction of a grooved portion in a balloon catheter according to a second cross-example, and FIG. 6B is a cross-sectional view showing an outer tube of the balloon catheter in a smashed state from the state illustrated in FIG. 6A.

FIG. 7A is a cross-sectional view taken in a direction orthogonal to an axial direction of a grooved portion of a balloon catheter according to a third cross-example, and FIG. 7B is a sectional view showing an outer tube of the balloon catheter in a smashed state from the state illustrated in FIG. 7A.

FIG. 8 is a partially-omitted lateral view showing a grooved portion in a balloon catheter according to a fourth cross-example.

FIG. 9 is a cross-sectional view taken in a direction orthogonal to an axial direction of a grooved portion of a balloon catheter according to a fifth cross-example.

FIG. 10 is a lateral cross-sectional view showing a modification example of a structure of a joint section between a balloon and an outer tube.

FIG. 11 is a cross-sectional view taken in a direction orthogonal to an axial direction of a grooved portion of a balloon catheter according to a sixth cross-example.

FIG. 12 is an enlarged lateral cross-sectional view showing a distal end side of a balloon catheter according to a second embodiment disclosed here.

FIG. 13A is a cross-sectional view of a known catheter taken in a direction orthogonal to an axial direction of an inflation lumen formed of an inner tube and an outer tube in a conventional configuration, and FIG. 13B is a cross-sectional view showing the outer tube in a smashed state from the state illustrated in FIG. 13A.

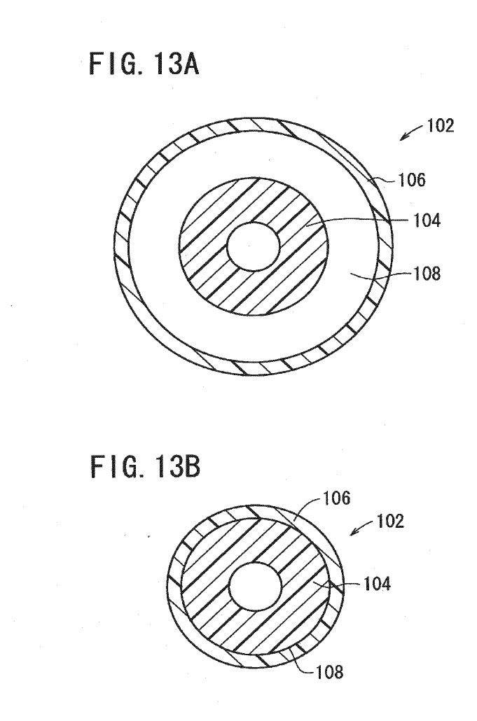

### DETAILED DESCRIPTION

Set forth below with reference to the accompanying drawing figures is a detailed description of examples of the inventive balloon catheter disclosed here.

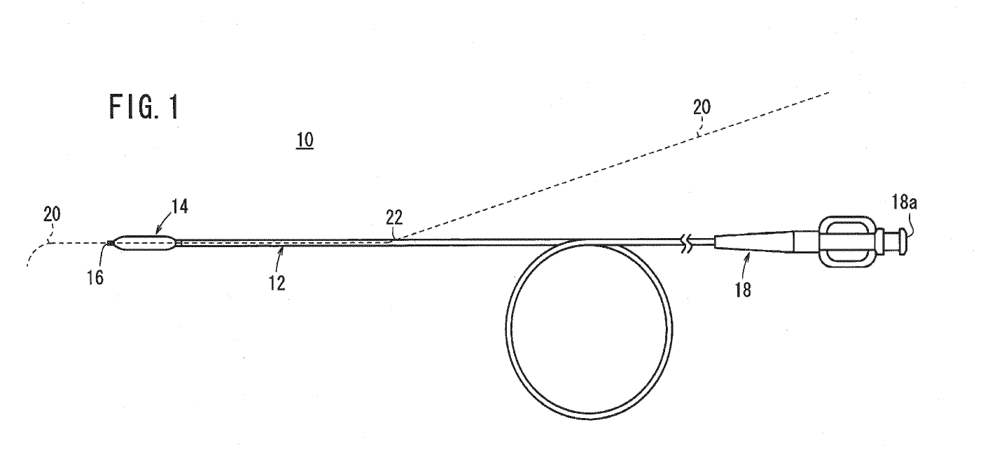

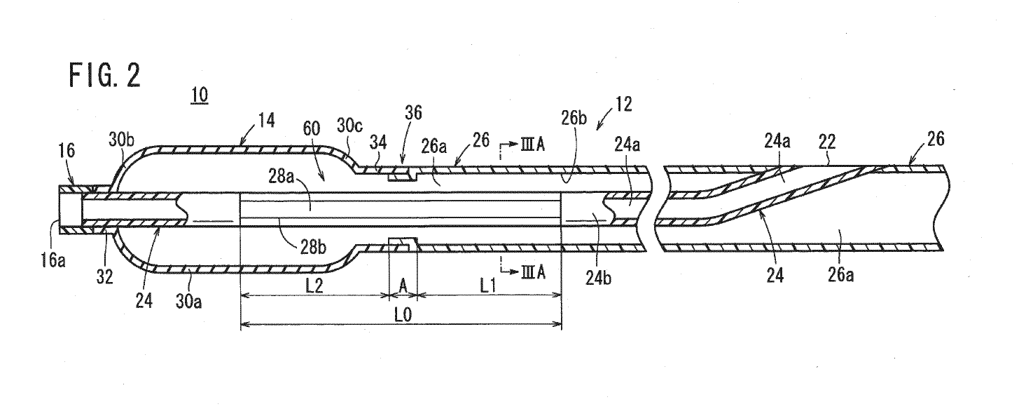

Referring initially to FIGS. 1 and 2, the balloon catheter 10 according to the present embodiment is a so-called PTCA (Percutaneous Transluminal Coronary Angioplasty) expansion catheter that includes an elongated shaft main body 12 configured to be inserted into a biological organ such as into a coronary artery and a balloon 14 disposed on the distal end and configured to be expanded at a stenosis (lesion) so that the stenosis is expanded and treated. The disclosure here is applicable not only to such a PTCA expansion catheter, but also to, for example, a catheter for improving a lesion formed in a biological organ such as other blood vessels, a bile duct, a trachea, an esophagus, a urethra or other organs.

As shown in FIGS. 1 and 2, the balloon catheter 10 includes the elongated shaft main body 12 having a small diameter, a distal end piece 16 fixed to the most distal end of the shaft main body 12, the balloon 14 disposed on a proximal end side of the distal end piece 16 and a hub 18 provided on a proximal end side of the shaft main body 12.

In the present embodiment, the balloon catheter 10 is of a type called a rapid exchange type catheter, in which an opening or open proximal end 22 through which a guide wire 20 is extracted is provided at a location slightly near to the distal end side of an intermediate portion of the shaft main body 12. However, the invention here can be applied also to any other kinds of balloon catheters such as a balloon catheter of an over-the-wire type in which an opening for a guide wire is provided in the hub 18 on the proximal end side of the shaft main body 12. In FIGS. 1 and 2, the right side (the hub 18 side) of the shaft main body 12 is referred to as the “proximal end” side (“rear end” side), and the left side (the distal end piece 16 and balloon 14 side) of the shaft main body 12 is referred to as “distal end” side. This applies also to the other figures.

Referring to FIG. 2, the shaft main body 12 includes: an inner tube 24 (also referred to as inner tube shaft or guide wire tube) in which a wire lumen 24a into which the guide wire 20 is to be fitted is formed; and an outer tube 26 (also referred to as outer tube shaft) cooperating with an outer circumferential surface of the inner tube 24 to define an inflation lumen 26a for supplying inflation fluid for inflating the balloon 14. A portion of the shaft main body 12 from the distal end to the opening 22 is thus formed as a coaxial double tube.

The inner tube 24 extends in the balloon 14 and the outer tube 26 has the distal end positioned substantially centrally of the distal end piece 16 and has the proximal end joined in a liquid-tight manner to the opening 22 formed at an intermediate portion of the outer tube 26. Accordingly, the guide wire 20 inserted into the entrance provided by a distal end opening 16a of the distal end piece 16 advances from the distal end side toward the proximal end side in the wire lumen 24a of the inner tube 24 and is extracted from the opening 22 serving as an exit.

The outer tube 26 extends from the proximal end of the balloon 14 to the distal end of the hub 18 so that a double tube exists which forms the inflation lumen 26a between the outer tube 26 and the inner tube 24 in a portion from the distal end of the outer tube 26 to the opening 22, while a portion from the opening 22 to the hub 18 forms a single pipe. The outer tube 26 can deliver inflation fluid, which is fed under pressure from a luer taper 18a or the like provided in the hub 18 to the balloon 14 by a pressure application apparatus such as an indeflator.

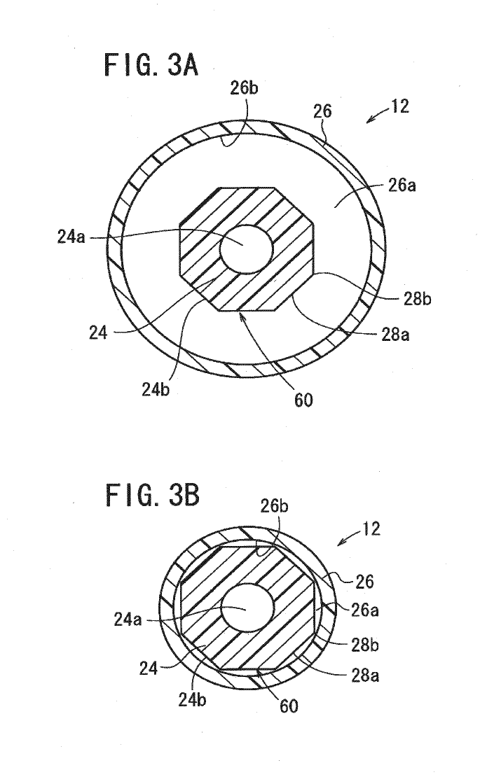

As shown in FIGS. 2 and 3A, the inner tube 24 is generally a cylindrical tube, and the inflation lumen 26a of an annular shape is located between the outer surface 24b of the inner tube 24 and the inner surface 26b of the outer tube 26. The outer surface 24b of the inner tube 24 has, at least over an axially extending portion of the outer surface 24b of the inner tube 24 (a portion represented by the distance L0 in FIG. 2), a non-circular region 60 whose cross section taken in a direction perpendicular to the axial direction of the inner tube 24 has a non-circular shape. The non-circular region 60 or shape used here refers to a non-circular region or shape when no external or internal force is applied to the catheter, that is when the catheter is lying at rest before being used. FIG. 3A shows that the shape of the outer surface of the inner tube 24 in the non-circular region 60 is a polygonal shape (for example, an octagonal shape in FIG. 3A). The non-circular region 60 includes axially extending grooved portions 28a and axially extending projecting portions 28b, wherein the grooved portions 28a and the projecting portions 28b circumferentially alternate with one another.

When the balloon catheter 10 is operated in a human body, even when the shaft main body 12 is elongated to reduce the diameter of the shaft main body by tensile force, bending force or the like applied from the proximal end side until the outer surface 24b of the inner tube 24 and the inner surface 26b of the outer tube 26 are brought into contact with each other as shown in FIG. 3B, the non-circular region 60 between the grooved portions 28a and the projecting portions 28b prevents the inflation lumen 26a from being completely blocked or closed-off, and provides a plurality of auxiliary lumens (circumferentially spaced apart auxiliary lumens) that assure the flow of the inflation fluid.

The projecting portions 28b are portions which serve, even in a state in which the outer surface 24b of the inner tube 24 and the inner surface 26b of the outer tube 26 contact each other as shown in FIG. 3B, as a support post between the outer surface 24b and the inner surface 26b to assure a space therearound in order to prevent the inflation lumen 26a from being fully blocked. The distal end of the projecting portions 28b need not have an angular shape, but can instead possess a curved shape or the like. The projecting portions 28b may be otherwise represented, for example, as support posts, angular portions, ridge portions or flow path holding portions, and this similarly also applies to modified examples and other embodiments of the balloon catheter such as those described below. Further, the grooved portions 28a are portions which serve as flow paths (groove-shaped flow paths) between the outer surface 24b and the inner surface 26b in order to prevent, even in a state in which the outer surface 24b and the inner surface 26b contact each other, the inflation lumen 26a from being blocked. The grooved portions 28a need not possess a flattened surface as shown in FIGS. 3A and 3B, but can possess a somewhat concaved (curved) shape or the like. From a relationship with the projecting portions 28b, the grooved portions 28a can be represented also as flattened portions, valley portions or flow path holding portions. This similarly also applies to modified examples and other embodiments of the balloon catheter such as those described below.

The inner tube 24 is a tube having, for example, an outer diameter of approximately 0.1 mm to 1 mm, preferably approximately 0.3 mm to 0.7 mm, a thickness of approximately 10 μm to 150 μm, preferably approximately 20 μm to 100 μm, and a length of approximately 100 mm to 2,000 mm, preferably approximately 150 mm to 1,500 mm. The grooved portions 28a and the projecting portions 28b which form auxiliary lumens while preventing blocking of the inflation lumen 26a may be formed by increasing or decreasing the thickness of the inner tube 24, for example, within a range of approximately 8 μm to 120 μm.

The outer tube 26 is a tube having, for example, an outer diameter of approximately 0.3 mm to 3 mm, preferably of approximately 0.5 mm to 1.5 mm; a thickness of approximately 10 μm to 150 μm, preferably of approximately 20 μm to 100 μm; and a length of approximately 300 mm to 2,000 mm, preferably of approximately 700 mm to 1,600 mm.

Preferably, the inner tube 24 and the outer tube 26 are structured such that they have a suitable flexibility and a suitable strength (resilience or rigidity) in order that, while an operator grips and operates the proximal end side of the balloon catheter 10, the elongated shaft main body 12 can be inserted relatively smoothly into a biological organ such as a blood vessel. Therefore, the inner tube 24 and the outer tube 26 may be formed for example from one of, a mixture of or a multilayer tube having two or more of polymeric materials such as polyolefin (polyethylene, polypropylene, polybutene, ethylene-propylene copolymer, ethylene-vinyl acetate copolymer, ionomer, or a mixture of two or more of the above materials, for example), polyvinylchloride, polyamide, polyamide elastomer, polyurethane, polyurethane elastomer, polyimide, fluorocarbon resin or the like.

The method of forming or producing the grooved portions 28a and the projecting portions 28b on the inner tube 24 (groove working method) is not limited particularly, but examples include configuring the mold exit for extrusion molding of a tube in a polygonal shape, forming the grooved portions 28a and the projecting portions 28b on the outer surface of a cylindrical tube using a laser or by melting, cutting or the like.

The balloon 14 can be contracted (folded) and expanded by varying the internal pressure. As shown in FIG. 2, the balloon 14 includes a tubular portion 30a which is expanded tubularly (cylindrically) by inflation fluid injected into the inside of the balloon 14 through the inflation lumen 26a, a distal end tapering portion 30b having a diameter gradually decreasing toward the distal end side of the tubular portion 30a, and a proximal end tapering portion 30c having a diameter gradually decreasing toward the proximal end side of the tubular portion 30a.

In the illustrated embodiment, the balloon 14: is joined, at a cylindrical distal end side non-expansion portion 32 provided on the distal end side of the distal end tapering portion 30b, in a liquid-tight manner to the outer circumferential surface of the inner tube 24; and is joined in the illustrated embodiment, at a cylindrical proximal end side non-expansion portion 34 provided on the proximal end side of the proximal end tapering portion 30c, in a liquid-tight manner to the distal end portion of the outer tube 26 so that the balloon 14 is securely mounted on the shaft main body 12. The inner diameter of the distal end side non-expansion portion 32 substantially coincides with the outer diameter of the inner tube 24, and the outer diameter of the proximal end side non-expansion portion 34 substantially coincides with the outer diameter of the outer tube 26. The balloon 14, the inner tube 24 and the outer tube 26 have only to be securely mounted in a liquid-tight state and may be joined together, for example, by adhesion or heat seal.

The size of the balloon 14 upon expansion is such that, for example, the outer diameter of the tubular portion 30a is approximately 1 mm to 6 mm, preferably approximately 1 mm to 5 mm, and the length is approximately 5 mm to 50 mm, preferably approximately 5 mm to 40 mm. Further, for example, the outer diameter of the distal end side non-expansion portion 32 is approximately 0.5 mm to 1.5 mm, preferably approximately 0.6 mm to 1.3 mm and is substantially equal to the outer diameter of the distal end piece 16, and the length is approximately 1 mm to 5 mm, preferably approximately 1 mm to 3 mm. For example, the outer diameter of the proximal end side non-expansion portion 34 is approximately 0.5 mm to 1.6 mm, preferably approximately 0.7 mm to 1.5 mm, and the length is approximately 1 mm to 5 mm, preferably approximately 2 mm to 4 mm. Further, for example, the length of each of the distal end tapering portion 30b and the proximal end tapering portion 30c is approximately 1 mm to 10 mm, preferably approximately 3 mm to 7 mm. For such a balloon 14 as described above, a suitable flexibility is required similarly to the inner tube 24 and the outer tube 26, and strength sufficient to expand a stenosis with certainty is also required. The material of the balloon 14 may be, for example, the same as that of the inner tube 24 and the outer tube 26 described by way of example above, but may naturally be some other material as well.

As shown in FIG. 2, a joint section 36 (axial overlap) between the proximal end side of the balloon 14 (the proximal end side non-expansion portion 34) and the outer tube 26 extends by a predetermined distance (a distance A) in the axial direction. Further, the inner diameter of the joint section 36 has a tendency to be smaller than that of any other portion of the proximal end side non-expansion portion 34 and the outer tube 26. Accordingly, a portion of the inflation lumen 26a which corresponds to the joint section 36 is generally narrower than any other portion.

The distal end piece 16 is a short tube having an outer diameter substantially equal to that of the distal end side non-expansion portion 32 of the balloon 14 and having an inner diameter substantially equal to the outer diameter of the inner tube 24. In the illustrated embodiment, the proximal end of the distal end piece 16 abuts the distal end of the of the non-expansion portion 32 of the balloon 14 so that there is no axial overlap between the two. By way of example, the length of the distal end piece 16 in the axial direction is approximately 0.5 mm to 20 mm. The distal end piece 16 is fitted externally with and joined in a liquid-tight manner to the distal end portion of the inner tube 24 and projects distally beyond the distal end opening of the wire lumen 24a. Further, the distal end piece 16 is joined at its proximal end face to the distal end face of the distal end side non-expansion portion 32 of the balloon 14. As recognized from FIG. 2, the distal end opening 16a of the distal end piece 16 communicates with the wire lumen 24a of the inner tube 24 and serves as an entrance for the guide wire 20.

The distal end piece 16 is suitably configured in regard to the material and the shape so that it is more flexible at least than the inner tube 24 or the outer tube 26, and may be formed for example from one of, a mixture of or a multilayer tube having two or more of polymeric materials such as polyolefin (polyethylene, polypropylene, polybutene, ethylene-propylene copolymer, ethylene-vinyl acetate copolymer, ionomer, or a mixture of two or more of the above materials, for example), polyvinylchloride, polyamide, polyamide elastomer, polyurethane, polyurethane elastomer, polyimide, fluorocarbon resin or the like. Such a distal end piece 16 as described above is a portion which advances flexibly as the most distal end of the balloon catheter 10 along a curved portion, a concave portion, a convex portion and so forth in a biological organ and penetrates through a stenosis (lesion) to lead relatively smooth insertion of the balloon catheter 10. It is to be noted that the distal end piece 16 may be omitted. In this instance, the balloon catheter 10 may be configured such that the most distal end position of the inner tube 24 and the most distal end position of the distal end side non-expansion portion 32 of the balloon 14 coincide with each other or such that the most distal end position of the inner tube 24 projects a little distally beyond the most distal end position of the distal end side non-expansion portion 32.

The operation of the balloon catheter 10 according to this embodiment configured as above is now set forth.

First, the form of a stenosis (lesion) appearing in a coronary artery or the like is specified by angiography or intravascular ultrasonography. Then, for example by Seldinger method, the guide wire 20 is precedently introduced percutaneously into the blood vessel from the thigh or the like. Then, the guide wire 20 is inserted from the distal end opening 16a of the distal end piece 16 and then through the wire lumen 24a of the inner tube 24 toward the opening 22 while the balloon catheter 10 is inserted into the coronary artery. Then, under radioscopy, the guide wire 20 is advanced to the target stenosis and is further advanced to pass through the stenosis and indwelled while the balloon catheter 10 is advanced into the coronary artery along the guide wire 20.

Then, when the distal end piece 16 of the balloon catheter 10 reaches and passes the stenosis, the balloon 14 is positioned at the stenosis, and so inflation fluid such as contrast agent is pressure fed from the hub 18 side into the inflation lumen 26a, whereby the balloon 14 is expanded to expand the stenosis. Consequently, predetermined medical treatment can be carried out. After the medical treatment, the inflation fluid is sucked out from within the balloon 14 to the hub 18 side through the inflation lumen 26a so that the balloon 14 is contracted, and then the balloon catheter 10 is removed out of the body.

In this manner, expansion and contraction of the balloon 14 are carried out by circulating the inflation fluid through the inflation lumen 26a. Accordingly, when the balloon catheter 10 is advanced into the body, for example, if the distal end of the balloon catheter 10 is caught by the stenosis or a bent portion and the operator thus tries to pull the shaft main body 12 to the proximal end side or the like, the inner tube 24 or the outer tube 26 may be elongated. If this actually occurs, the outer surface 24b of the inner tube 24 and the inner surface 26b of the outer tube 26 may be brought into contact with each other or excessively approach each other, whereby the inflation lumen 26a between the outer surface 24b of the inner tube 24 and the inner surface 26b of the outer tube 26 becomes narrow and, depending on the circumstances, becomes fully blocked. In that case, smooth circulation of the inflation fluid is obstructed, resulting in the possibility that expansion or contraction of the balloon 14 may become difficult.

Particularly, the joint section 36 between the proximal end side of the balloon 14 and the outer tube 26 has an inner diameter smaller than that of any other portion of the proximal end side non-expansion portion 34 and the outer tube 26, and the portion of the inflation lumen 26a corresponding to the joint section 36 is configured narrower than other portions. Therefore, if such elongation as described above occurs, then the possibility that the inflation lumen 26a may be blocked in the proximity of the joint section 36 is higher than that at any other portion.

Therefore, in the balloon catheter 10 according to the present embodiment, as shown in FIG. 3A, since the grooved portions 28a and the projecting portions 28b are provided as the non-circular region 60 on the outer surface 24b of the inner tube 24, even when the outer surface 24b of the inner tube 24 and the inner surface 26b of the outer tube 26 are brought into contact with each other, the inner tube 24 and the outer tube 26 are prevented from closely contacting each other over the whole circumference by the grooved portions 28a and the projecting portions 28b as shown in FIG. 3B. Therefore, circulation of the inflation fluid through the inflation lumen 26a is assured with certainty, and such a situation that expansion and contraction of the balloon 14 become difficult can be avoided or prevented.

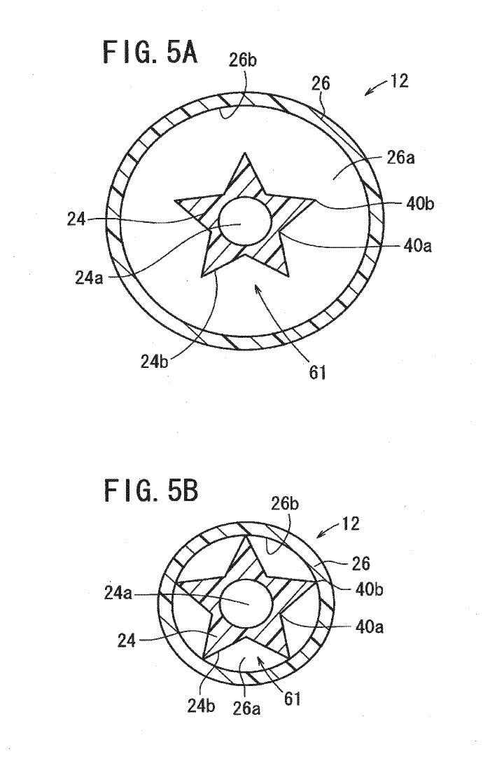

In particular, since the non-circular region 60 is provided on the outer surface 24b, that is, provided on one side between the outer surface 24b and the inner surface 26b which configure the inflation lumen 26a, even when the outer surface 24b and the inner surface 26b are brought into contact with each other, the inflation lumen 26a can be prevented from being blocked. It is to be noted that the non-circular shape may be such that a sectional shape of at least one of the outer surface 24b and the inner surface 26b forming the inflation lumen 26a is any other shape than a circular shape and that, when the outer surface 24b and the inner surface 26b are brought into contact with each other, a space to function as a flow path can be ensured. Examples include a polygonal shape as described hereinabove (FIG. 3A), a star shape (FIG. 5A) or other shapes. In other words, cross sections of the outer surface 24b of the inner tube 24 and the inner surface 26b of the outer tube 26 which define the inflation lumen 26a may have any shape that are not analogous to each other (non-analogous shapes or shapes that are not the same as one another). Or, even if the cross sections have analogous shapes (i.e., the same shape), the flow paths can be prevented from being blocked if the phases of the cross-sectional shapes are displaced from each other. The configuration of the outer surface 24b of the inner tube 24 and the inner surface 26b of the outer tube 26 is such that the contact regions between the outer surface 24b of the inner tube 24 and the inner surface 26b of the outer tube 26 are circumferentially spaced apart from one another so that one or more axially extending spaces (axially extending non-contact regions) exist between the outer surface 24b of the inner tube 24 and the inner surface 26b of the outer tube 26. The number of such grooved portions 28a or projecting portions 28b which configure the non-circular region 60 may be one or more in a circumferential direction of the outer surface 24b.

As recognized from FIG. 3A, it can be sufficiently achieved to prevent the flow path from being blocked if at least one of the grooved portions 28a and the projecting portions 28b which configure the non-circular region 60 is provided. Further, even if only the projecting portions 28b are provided, since side portions of the projecting portions 28b are more concaved compared to the projecting portions 28b, substantially grooved portions are formed. Similarly, even if only the grooved portions 28a are provided, since edge portions of the grooved portions 28a are more projecting compared to the bottom of the grooved portions 28a, substantially projecting portions are formed.

As shown in FIG. 2, the non-circular region 60 comprising the grooved portions 28a and the projecting portions 28b is provided over the distance L0 including the joint section 36 indicated by the distance A in an extension direction (in the axial direction) of the inner tube 24, or in other words, in the extension direction of the inflation lumen 26a. In this manner, the non-circular region 60 is provided so as to extend forwardly and backwardly beyond the range of the joint section 36 from the distal end side to the proximal end side of the joint section 36. Accordingly, since the non-circular region 60 is disposed at a portion corresponding to the joint section 36 estimated to be most likely to be blocked within the entire inflation lumen 26a, the inflation lumen 26a can be prevented from being blocked with a higher degree of certainty.

Because the shaft main body 12 is frequently pulled to the proximal end side by an operator, it is estimated that the joint section 36 may block the inflation lumen 26a while being elongated in the direction toward the proximal end. Therefore, preferably the non-circular region 60 at least includes the joint section 36 and is provided at a position displaced by a predetermined distance toward the proximal end side from the joint section 36, that is at an area indicated by the distance A and a distance L1 in FIG. 2. The non-circular region 60 of the inner tube 24 thus axially overlaps, or is axially coextensive with, the joint section 36 and a portion of the outer tube extending proximally by a distance L1.

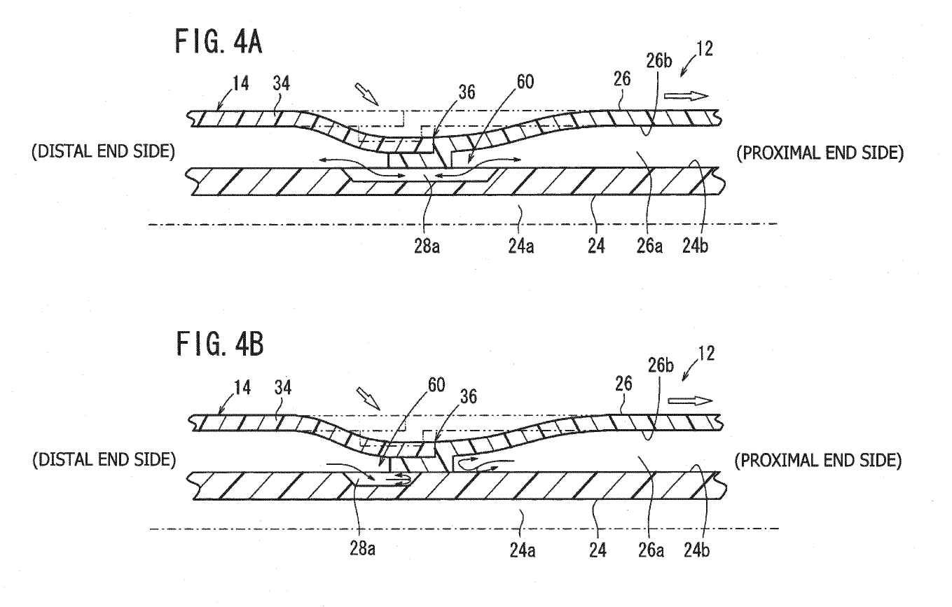

Consequently, as shown in FIG. 4A, the inflation lumen 26a is ensured with certainty by the non-circular region 60 (the grooved portions 28a), and relatively smooth circulation of the inflation fluid can be ensured.

In a situation in which the non-circular region 60 does not extend to the proximal end side farther than the joint section 36 as shown in FIG. 4B, for example in the case in which the proximal end side of the grooved portions 28a coincides with the proximal end side of the joint section 36, the joint section 36 is displaced toward the proximal end side by elongation of the shaft main body 12 due to the tensile force toward the proximal end side and is thus brought into contact with the outer surface 24b of the inner tube 24, resulting in the possibility that the assurance of the inflation lumen 26a by the grooved portions 28a may become difficult. Accordingly, it is effective to assure the existence of the inflation lumen 26a to configure the non-circular region 60 so as to be axially coextensive with the joint section 36 and to also extend proximally beyond the proximal end of the joint section 36.

Naturally, as shown in FIG. 2, the non-circular region 60 may extend distally beyond the distal end of the joint section 36 represented by the distance L2. If the non-circular region 60 extends to the expansion area of the balloon 14 in this manner, the inflation lumen 26a can be prevented from being blocked with a higher degree of certainty. In other words, the non-circular region 60 may be provided over the whole length of the inflation lumen 26a, that is, may be provided from the most distal end to the most proximal end (to the opening 22) of the inner tube 24. When the balloon catheter is of the over-the-wire type, grooved portions may be provided over the whole inner tube which extends from the distal end to the hub at the proximal end of the balloon catheter. However, if the non-circular region 60 extends over the whole inner tube 24 or over most part of the inner tube 24 in this manner, then the entire inner tube 24 becomes flexible, resulting in the possibility that also the rigidity of the proximal end side of the shaft main body 12 may drop and the operability may be deteriorated. Therefore, in this instance, it is preferable to raise the rigidity particularly of the proximal end side portion of the outer tube 26.

The non-circular region 60 (the grooved portions 28a and the projecting portions 28b) which assists the function of the inflation lumen 26a may have a shape other than the polygonal shape shown in FIG. 3A. In short, the non-circular region 60 may be configured such that it is communicated in the axial direction over the distance L0 (refer to FIG. 2) as the range for providing the non-circular region 60.

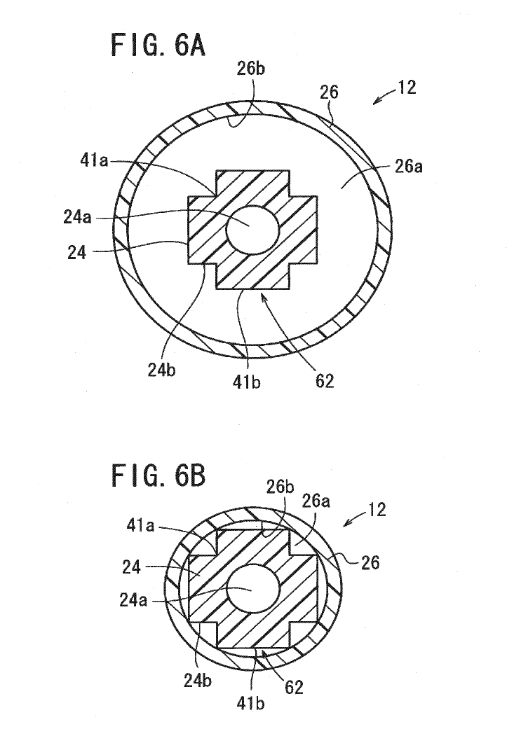

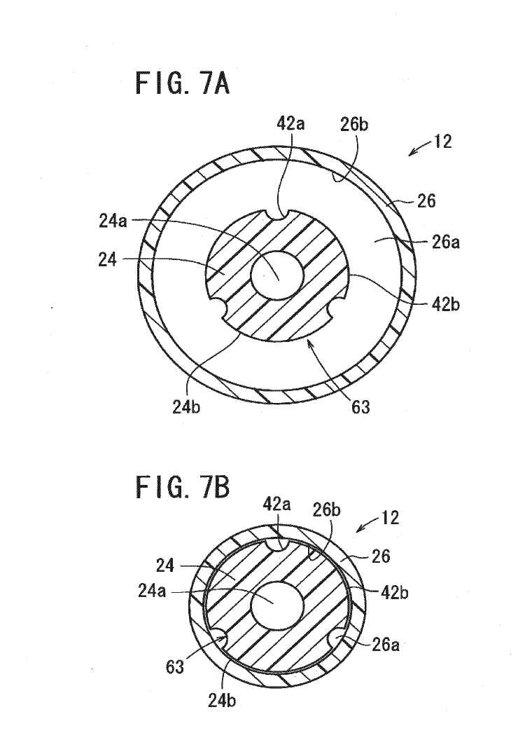

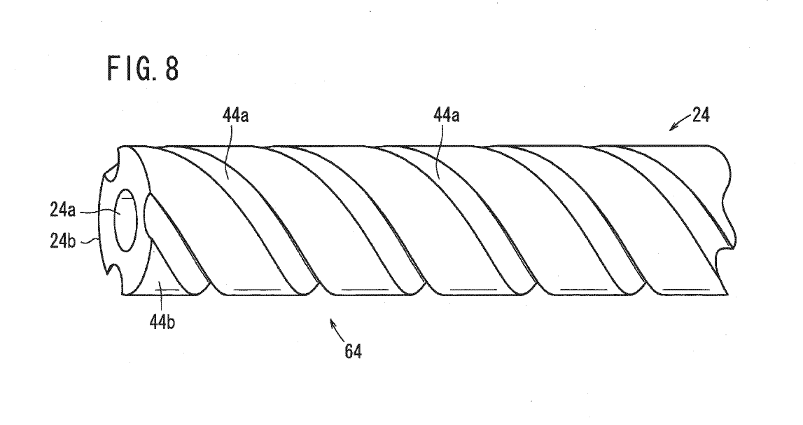

For example, as shown in FIGS. 5A and 5B, a star-shaped non-circular region 61 having a plurality of grooved portions (recessed portions, valley portions or flow path holding portions) 40a and a plurality of projecting portions (support posts, angular portions, ridge portions or flow path holding portions) 40b on the outer surface 24b of the inner tube 24 may be provided. Alternatively, as shown in FIGS. 6A and 6B, a cross-shaped non-circular region 62 having a plurality of grooved portions (recessed portions, valley portions or flow path holding portions) 41a and a plurality of projecting portions (support posts, angular portions or flow path holding portions) 41b on the outer surface 24b of the inner tube 24 may be provided. Further, as shown in FIGS. 7A and 7B, a non-circular region 63 circumferentially having one or a plurality of grooved portions (recessed portions, valley portions or flow path holding portions) 42a and one or a plurality of projecting portions (support posts or flow path holding portions) 42b extending linearly in the axial direction on the outer surface 24b of the inner tube 24 and further having one or a plurality of recessed portions as viewed in cross-section may be provided. Still further, as shown in FIG. 8, another possibility involves a non-circular region 64 having one or a plurality of spiral grooved portions (recessed portions, valley portions or flow path holding portions) 44a and one or a plurality of spiral projecting portions (support posts or flow path holding portions) 44b both of which extend in the axial direction while extending around the outer surface 24b of the inner tube 24 and are juxtaposed with each other.

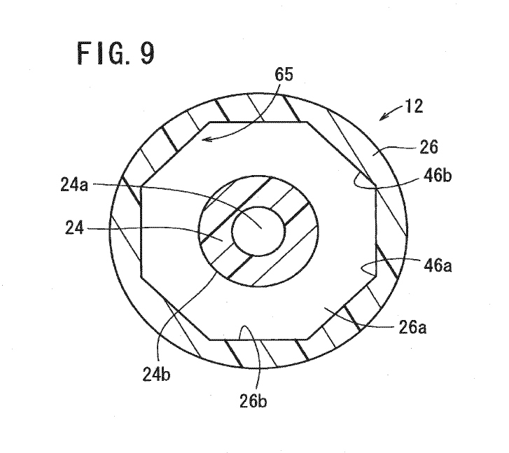

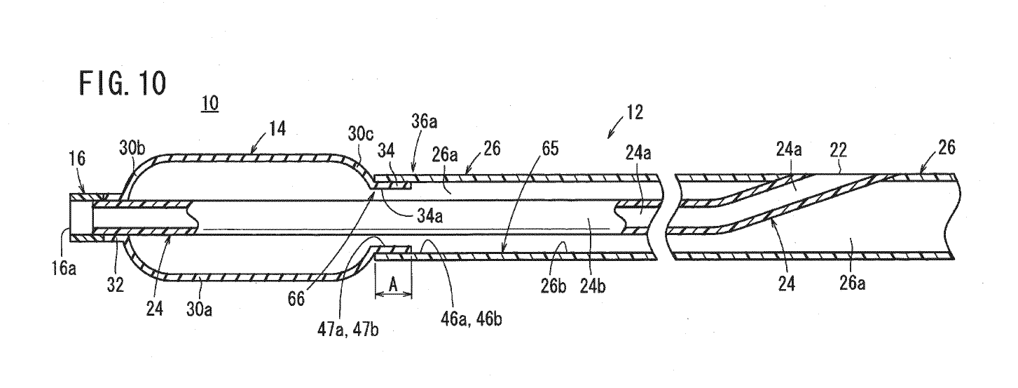

Such non-circular regions 60-64 as described above may be provided not on the inner tube 24 but, for example, as a non-circular region 65 having grooved portions (recessed portions, valley portions or flow path holding portions) 46a and projecting portions (support posts or flow path holding portions) 46b on the inner surface 26b of the outer tube 26 as shown in FIG. 9. The non-circular region 65 may be provided at an area of the inner surface 26b of the outer tube 26 at least including the joint section 36 indicated by the distance A in FIG. 2 and of course can be formed also on the proximal end side of the joint section 36. With this configuration, since the non-circular region 65 (the grooved portions 46a and the projecting portions 46b) is provided on the inner surface itself of the joint section 36 which may possibly block the inflation lumen 26a, the inflation lumen 26a can be prevented from being blocked in the proximity of the joint section 36 with a higher degree of accuracy.

Where the joint section between the balloon 14 and the outer tube 26 is a joint section 36a configured such that the proximal end side non-expansion portion 34 of the balloon 14 is joined so as to be received in the inside of the outer tube 26 as shown in FIG. 10, a non-circular region 66 may be formed on the inner surface 34a of the proximal end side non-expansion portion 34 of the balloon 14 which is positioned on the innermost side of the joint section 36a indicated by the distance A in FIG. 10. The non-circular region 66 may include grooved portions (recessed portions, valley portions or flow path holding portions) 47a and projecting portions (support posts or flow path holding portions) 47b respectively corresponding to the grooved portions 46a and the projecting portions 46b. In this state, the non-circular region 65 formed from the grooved portions 46a and the projecting portions 46b may be provided on the outer tube 26 (inner surface of the outer tube 26) on the proximal end side of the joint section 36a.

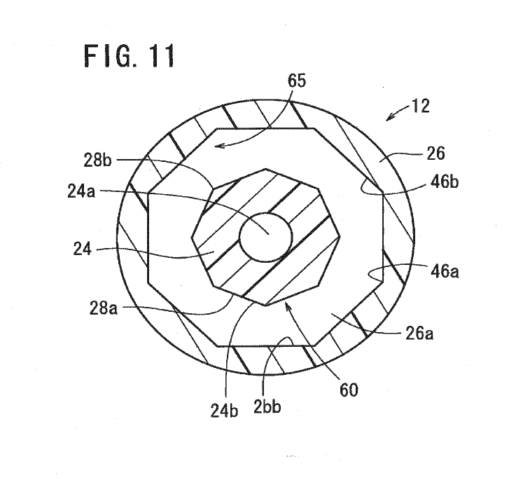

Further, the balloon catheter 10 may be configured such that, for example, the non-circular region 60 is provided on the outer surface 24b of the inner tube 24 and the non-circular region 65 is provided on the inner surface 26b of the outer tube 26 as shown in FIG. 11. Where a non-circular region (a grooved portion and a projecting portion) is provided on both the inner tube 24 and the outer tube 26 as shown in FIG. 11, it is preferable to utilize an arrangement or configuration in which the phases of the grooved portions 28a and 46a correspond to each other and the phases of the projecting portions 28b and 46b correspond to each other. Stated differently, when the non-circular region is constituted by a non-circular cross-sectional shape on the outer surface 24b of the inner tube 24 and the same non-circular cross-sectional shape on the inner surface 26b of the outer tube 26, the non-circular cross-sectional shape on the outer surface 24b of the inner tube 24 and the non-circular cross-sectional shape on the inner surface 26b of the outer tube 26 are rotationally offset from one another as shown in FIG. 11. This arrangement or configuration helps prevent the grooved portions and the projecting portions of the inner tube 24 and the outer tube 26 from engaging with each other, and the inflation lumen 26a can thus be prevented from being blocked.

In this manner, the non-circular region 60 or the like have only to be provided at a position in which the joint section 36 is included and the portion on the proximal end of the joint section 36 is covered on at least one of the inner surface of the inflation lumen 26a (the outer surface 24b of the inner tube 24) and the outer surface of the inflation lumen 26a (the inner surface 26b of the outer tube 26 or the inner surface 34a of the proximal end side non-expansion portion 34 of the balloon 14). Further, where the non-circular region 60 or the like is provided on the outer surface of the inflation lumen 26a, it is preferable to provide the non-circular region 60 or the like on the innermost inner surface facing the inner tube 24. That is, where the non-circular region 60 or the like is on the outer surface of the inflation lumen 26a, it is preferable to provide the non-circular region 60 or the like on the whichever of the inner surface 26b of the outer tube 26 and the inner surface 34a of the proximal end side non-expansion portion 34 of the balloon 14 is innermost (i.e., whichever of the surfaces is located most radially inward).

Further, with respect to the non-circular regions 60-66 described above, in the non-circular region 60 of a polygonal shape as shown in FIG. 3A, the non-circular region 61 of a star shape as shown in FIG. 5A, the non-circular region 62 of a cross shape as shown in FIG. 6A, the non-circular region 63 having grooved portions 42a extending linearly in the axial direction on the outer surface 24b of the inner tube 24 as shown in FIG. 7A and so forth, for example, even when the grooved portions 28a (projecting portions 28b) or the like is elongated by elongation of the shaft main body 12, the shape of the grooved portions is maintained and the inflation lumen 26a can be ensured with a relatively higher degree of certainty.

According to the various embodiments and modifications described above, a longitudinally extending portion of the inflation lumen is formed as a non-circular region in which: i) the cross-sectional shape (and size) of the inner surface cross-sectional shape of the outer tube differs from the cross-sectional shape (and size) of the outer surface cross-sectional shape of the inner tube (e.g., one of the surfaces is circular in cross-section while the other is non-circular such as shown in FIGS. 3 and 5-7); or ii) the size of the inner surface cross-sectional shape of the outer tube differs from the size of the outer surface cross-sectional shape of the inner tube, the shape of the inner surface cross-sectional shape of the outer tube is the same as the shape of the outer surface cross-sectional shape of the inner tube, and the inner surface cross-sectional shape of the outer tube is rotationally offset from the outer surface cross-sectional shape of the inner tube as shown in FIG. 11).

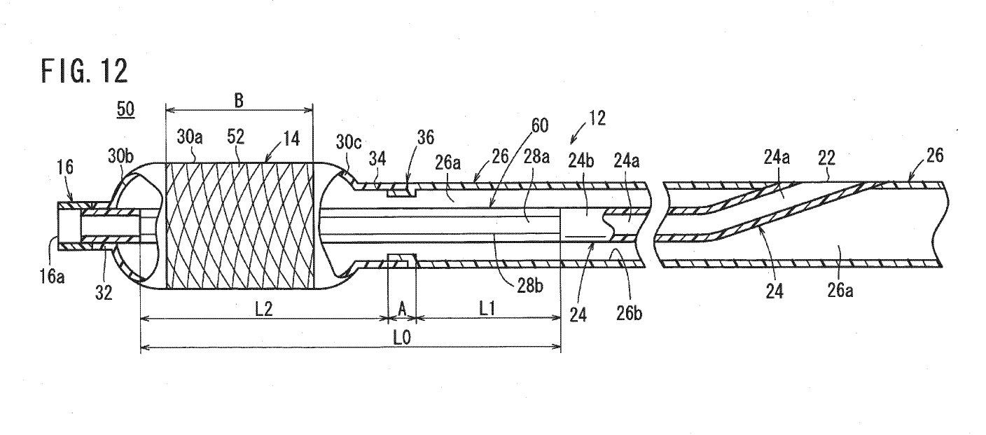

FIG. 12 is a lateral cross-sectional view showing, in an enlarged scale, the distal end side of a balloon catheter 50 according to a second embodiment of the present invention. In FIG. 12, the same reference symbols as those appearing in FIGS. 1-11 denote the same or equivalent elements, and a detailed description of such features is not repeated to avoid redundancy.

Referring to FIG. 12, the balloon catheter 50 according to the present embodiment is substantially the same as the balloon catheter 10 according to the first embodiment described above, except that the balloon catheter 50 has a stent 52 disposed on the outer periphery of the balloon 14 and is formed as a balloon catheter for stent delivery (stent system) which is used when the stent 52 is to be indwelled at the object lesion. In this instance, the balloon 14, a distal end side region of the outer tube 26 to which the proximal end side of the balloon 14 is joined or the like is acted upon by compulsory contraction by the compressive force of the stent 52. As a result, the outer tube 26 may be smashed and the inflation lumen 26a may be blocked, and there is the possibility that smooth expansion or contraction of the balloon 14 and uniform expansion of the stent 52 may become difficult.

Therefore, in the balloon catheter 50, each of the non-circular regions 60-64 described above is provided in a region at which the stent 52 is to be disposed, that is in a region to cover a position including a distance B from the distal end to the proximal end of the stent 52 as shown in FIG. 12 (the non-circular regions 60-64 axially overlap the region at which the stent 52 is located). With this configuration, relatively reliable and uniform expansion of the stent 52 can be ensured. Naturally, to ensure reliable expansion and contraction of the balloon 14, it is preferable to provide the non-circular region 60 or the like also for the distance A corresponding to the joint section 36 and the distances L1 and L2.

The detailed description above describes a catheter according to several embodiments and modifications disclosed by way of example. The invention here is not limited, however, to the precise embodiments and variations described above and illustrated in the drawing figures. Various changes, modifications and equivalents could be effected by one skilled in the art without departing from the spirit and scope of the invention as defined in the appended claims. It is expressly intended that all such changes, modifications and equivalents which fall within the scope of the claims are embraced by the claims.

What is claimed is:

1. A balloon catheter comprising:
an elongated outer tube extending in a longitudinal direction and possessing a longitudinal axis and a distal end portion, a proximal end portion and an intermediate portion located between the distal end portion and the proximal end portion, the outer tube possessing an inner surface;
an inner tube located inside the outer tube and possessing an outer surface and a distal end portion, the inner tube possessing an open distal end, an open proximal end and a lumen extending between the open distal end and the open proximal end, the open proximal end of the inner tube communicating outside the outer tube at the intermediate portion of the outer tube, the lumen in the inner tube being configured to receive a guide wire extending along the lumen and passing through the open distal end and the open proximal end;
a balloon possessing a distal end portion fixed to the distal end portion of the inner tube and possessing a proximal end portion fixed to the distal end portion of the outer tube at an axially overlapping joint section at which the distal end portion of the outer tube and the proximal end portion of the balloon axially overlap each other, the balloon being configured to be expanded upon introducing an inflation fluid into the balloon and being contractable when the inflation fluid is expelled from the balloon;
an inflation lumen between the inner surface of the outer tube and the outer surface of the inner tube to receive the inflation fluid to expand the balloon;
a longitudinally extending portion of the inflation lumen being a non-circular region at which at least one of the outer surface of the inner tube and the inner surface of the outer tube possesses a non-circular cross-sectional shape in a direction perpendicular to the longitudinal axis of the outer tube, the joint section being located in the non-circular region of the inflation lumen, and the non-circular region of the inflation lumen extending proximally beyond a proximal-most end of the axially extending joint section.

2. The balloon catheter according to claim 1, wherein the non-circular cross-sectional shape includes projecting portions formed on at least one of the outer surface of the inner tube and the inner surface of the outer tube to project toward the other one of the inner surface and the outer surface, the projecting portions extending in the longitudinally direction.

3. The balloon catheter according to claim 2, wherein the non-circular cross-sectional shape includes grooved portions on at least one of the outer surface of the inner tube and the inner surface of the outer tube, the grooved portions extending in the longitudinal direction.

4. The balloon catheter according to claim 1, wherein the non-circular cross-sectional shape includes grooved portions on at least one of the outer surface of the inner tube and the inner surface of the outer tube, the grooved portions extending in the longitudinal direction.

5. The balloon catheter according to claim 1, wherein the axially overlapping proximal end portion of the balloon and distal end portion of the outer tube each possess an inner surface, and the non-circular cross-sectional shape is on a radially innermost one of the inner surface of the axially overlapping proximal end portion of the balloon and the distal end portion of the outer tube.

6. The balloon catheter according to claim 1, wherein the non-circular region extends to a position which reaches an expansion portion of the balloon that expands outwardly upon introduction of the inflation fluid into the inflation lumen.

7. The balloon catheter according to claim 1, further comprising a stent disposed on an outer circumferential surface of the balloon, the non-circular region extending longitudinally to a position including a region axially overlapping with the stent.

8. The balloon catheter according to claim 1, wherein in the non-circular region, the cross-sectional shape of at least one of the outer surface of the inner tube and the inner surface of the outer tube in the direction perpendicular to the longitudinal axis of the outer tube is a polygonal cross-sectional shape, a star cross-sectional shape or a cross-sectional shape having one or more concave portions.

9. The balloon catheter according to claim 1, wherein the non-circular cross-sectional shape includes grooved portions extending in the longitudinal direction and each possessing a spiral shape.

10. A balloon catheter comprising:
an elongated outer tube extending in a longitudinal direction and possessing a longitudinal axis, the outer tube also possessing a distal end portion, a proximal end portion and an intermediate portion located between the distal end portion and the proximal end portion, the outer tube possessing an inner surface cross-sectional shape in a direction perpendicular to the longitudinal axis of the outer tube;
an inner tube located inside the outer tube and possessing a distal end portion and a proximal end portion, the outer tube also possessing an outer surface cross-sectional shape in a direction perpendicular to the longitudinal axis of the outer tube, the inner tube comprising an open distal end, an open proximal end and a lumen extending between the open distal end and the open proximal end, the proximal end portion of the inner tube passing through a tube wall of the outer tube at the intermediate portion of the outer tube so that the open proximal end of the inner tube communicates outside the outer tube at the intermediate portion of the outer tube, the lumen in the inner tube being configured to receive a guide wire extending along the lumen and passing through the open distal end and the open proximal end;
a balloon possessing a distal end portion fixed to the distal end portion of the inner tube and possessing a proximal end portion fixed to the distal end portion of the outer tube at an axially overlapping joint section at which the distal end portion of the outer tube and the proximal end portion of the balloon axially overlap each other, the balloon being configured to be expanded upon introducing an inflation fluid into the balloon and being contractable when the inflation fluid is expelled from the balloon;
an inflation lumen between the inner surface of the outer tube and the outer surface of the inner tube to receive the inflation fluid to expand the balloon;
a longitudinally extending portion of the inflation lumen being a non-circular region at which: i) a size and a shape of the inner surface cross-sectional shape of the outer tube differ from a size and a shape of the outer surface cross-sectional shape of the inner tube; or ii) the size of the inner surface cross-sectional shape of the outer tube differs from the size of the outer surface cross-sectional shape of the inner tube, the shape of the inner surface cross-sectional shape of the outer tube is the same as the shape of the outer surface cross-sectional shape of the inner tube, and the inner surface cross-sectional shape of the outer tube is rotationally offset from the outer surface cross-sectional shape of the inner tube; and
the joint section being located in the non-circular region of the inflation lumen, and the non-circular region of the inflation lumen extending proximally beyond a proximal-most end of the axially extending joint section.

11. A balloon catheter comprising:
an inner tube configured to receive a guide wire;
an outer tube disposed on an outer surface side of the inner tube and cooperating with the inner tube to define an inflation lumen between the inner tube and the outer tube;
a balloon possessing a distal end portion joined to the inner tube and possessing a proximal end portion joined to the outer tube, the balloon being expandable by introduction of inflation fluid into the balloon from the inflation lumen,
at least one of an outer surface of the inner tube and an inner surface of the outer tube is a non-circular region having a non-circular cross-sectional shape in a direction orthogonal to an extending direction of the inner tube;
the non-circular region being located at least in a joint section at which the balloon and the outer tube are joined together and extending to a position on a proximal side of the joint section.

12. The balloon catheter according to claim 11, wherein the non-circular region comprises projecting portions formed on at least one of the outer surface of the inner tube and the inner surface of the outer tube so as to project toward the other one of the inner surface of the outer tube and the outer surface of the inner tube, the projecting portions extending in an extending direction of the inner tube.

13. The balloon catheter according to claim 11, wherein the non-circular region comprises grooved portions formed on at least one of the outer surface of the inner tube and the inner surface of the outer tube, the grooved portions extending in an extending direction of the inner tube.

14. The balloon catheter according to claim 11, wherein the balloon and the outer tube each include an inner surface in the joint section, the non-circular region being on an innermost one of the inner surface of the balloon and the inner surface of the outer tube.

15. The balloon catheter according to claim 11, wherein the non-circular region extends to a position axially overlapping with an expansion portion of the balloon which expands upon introduction of the inflation fluid into the balloon.

16. The balloon catheter according to claim 11, further comprising a stent disposed on an outer circumferential surface of the balloon, the non-circular region axially overlapping a region at which the stent is disposed.

17. The balloon catheter according to claim 11, wherein, in the non-circular region, the cross-sectional shape of at least one of the outer surface of the inner tube and the inner surface of the outer tube in a direction orthogonal to the extending direction of the inner tube is a polygonal cross-sectional shape, a star cross-sectional shape or a cross-sectional shape having one or more concave portions.

18. The balloon catheter according to claim 11, wherein the non-circular region comprises spiral-shaped grooved portions on at least one of the outer surface of the inner tube and the inner surface of the outer tube, the spiral-shaped grooved portions extending in an extending direction of the inner tube.
